(19) United States
(12) Patent Application Publication
(10) Pub. No.: US 2014/0044687 A1
(43) Pub. Date: Feb. 13, 2014

(54) METHOD FOR USING REGULATORY T CELLS IN THERAPY

(71) Applicants: Miguel Forte, Rixensart (BE); Arnaud Foussat, Biot (FR)

(72) Inventors: Miguel Forte, Rixensart (BE); Arnaud Foussat, Biot (FR)

(73) Assignee: TXCELL, Valbonne (FR)

(21) Appl. No.: 14/007,441

(22) PCT Filed: Apr. 26, 2011
(86) PCT No.: PCT/IB2011/001283

§ 371 Date: Oct. 22, 2013

Related U.S. Application Data
(60) Provisional application No. 61/467,568, filed on Mar. 25, 2011.

(51) Int. Cl. A61K 35/14

(52) U.S. Cl. CPC A61K 35/17
USPC 424/93.71; 435/372.3

(57) ABSTRACT

A method of regulatory cell therapy for a treating a patient in need thereof, wherein 10⁴ to 10⁶ regulatory T cells are administrated to the patient.

### FIELD OF INVENTION

The present invention relates to regulatory T cells and their use in cell therapy for treating autoimmune diseases, inflammatory diseases, allergic or asthmatic condition, graft versus host disease or for preventing graft rejection.

### BACKGROUND OF INVENTION

The immune system is a complicated network of many different players which interact with each other and cooperate to protect against diseases and fight established diseases. Among these players are regulatory T cells which act to suppress immune activation and thereby maintain immune homeostasis and tolerance to self-antigens.

Regulatory T cells are described in the art to comprise distinct cell populations such as natural regulatory T cells (nTreg), type 1 regulatory T cells (Tr1) and Th3 cells.

Although the therapeutic potential of regulatory T cells was envisioned decades ago, clinical implementation of their potent immune regulatory activity by in vivo administration of agents has proven challenging. Adoptive regulatory T cells therapy is an attractive alternative to harness the immune suppressive activity of regulatory T cells. In this approach, regulatory T cells are isolated from a patient or a healthy donor, enriched, sometimes further expanded ex vivo, and re-infused either to the same patient or to allogeneic recipients.

Their use in treatment applications is problematic because they are present as only a very small percentage, approximately 1 to 5%, of human peripheral blood mononuclear cells. Thus, methods of activating and expanding, or inducing proliferation of regulatory T cells ex-vivo have been developed for use in the treatment of certain diseases. All these methods aimed at providing large numbers of regulatory T cells such as at least 10⁷ to 10⁹ cells to be re-infused into a patient in need thereof.

Indeed, one dogma of cell therapy relies on more cells to be infused, the better the effect such as seen for classical pharmaceutical compounds. As infused cells are less toxic for the patient as a chemical compound may be, great numbers of cells (10⁸-10¹⁰ cells) are generally administrated to the patients.

Another reason for administering great numbers of cells is that a large number of infused cells generally migrate preferably to liver, spleen and lungs and in a lower manner to the site of interest that can be any other part of the body.

Finally, regarding regulatory T cell therapy, clinicians are willing to achieve a high regulatory T cells: conventional T cells ratio and therefore administrate also for this reason a high number of cells to the patient.

While carrying out regulatory T cell therapy in a clinical trial, the inventors found that such high doses of regulatory T cells to be administrated in a patient were not as effective as it was thought in the art.

Therefore, there is a need for a new method for regulatory T cell therapy that is more efficient for treating a patient in need thereof.

### SUMMARY

One object of the invention is a method for treating a patient in need thereof, comprising administering to the patient a therapeutically effective dose of 10⁴ to 10⁶ regulatory T cells.

One object of the invention is regulatory T cells for use in or for treating an inflammatory or autoimmune condition a patient in need thereof, wherein a therapeutically effective dose of 10⁴ to 10⁶ regulatory T cells is to be administered to the patient.

In one embodiment of the invention, the regulatory T cells are autologous.

In another embodiment of the invention, the regulatory T cells are allogeneic.

In another embodiment of the invention, the regulatory T cells are polyclonal.

In another embodiment of the invention, the regulatory T cells are monoclonal.

In another embodiment of the invention, the regulatory T cells are specific for a single antigen. In another embodiment of the invention, the regulatory T cells are specific for multiple antigens. In another embodiment of the invention, the patient to be treated is suffering from an autoimmune disease, an inflammatory disease, an asthmatic or allergic condition, graft-versus-host disease or is undergoing a transplantation.

### DEFINITIONS

The term “antigen” as used herein refers to a protein, a peptide, or a lipid or a glycolipid compound to which the cells of this invention are being directed. In one embodiment, the term “antigen” may refer to a synthetically derived molecule, or a naturally derived molecule, which shares sequence homology with an antigen of interest, or structural homology with an antigen of interest, or a combination thereof. In one embodiment, the antigen may be a mimetope, wherein a “mimetope” is an amino acid sequence that mimics a native antigen and is immunogenic, inducing antibody that has the same biological activity as that induced by the native antigen. A “fragment” of the antigen refers to any subset of the antigen, as a shorter peptide or lipid. A “variant” of the antigen refers to a molecule substantially similar to either the entire antigen or a fragment thereof. Variant antigens may be conveniently prepared by direct chemical synthesis of the variant peptide or lipid compound, using methods well-known in the art.

The term “patient” as used herein refers to a human being.

The term “effective amount” as used herein refers to an amount sufficient to cause a beneficial or desired clinical result (e.g. improvement in clinical condition).

The term “clone” or “clone population” as used herein refers to a population of differentiated cells being derived from a unique differentiated cell.

The term “treatment” as used herein refers to clinical intervention in an attempt to alter the natural course of a disease of the subject to be treated, and may be performed either for prophylaxis or during the course of clinical pathology. Desirable effects include, but are not limited to, preventing occurrence or recurrence of disease, alleviating symptoms, suppressing, diminishing or inhibiting any direct or indirect pathological consequences of the disease, lowering the rate of disease progression, ameliorating or palliating the disease state, and causing remission, maintaining remission state or improving prognosis. Regulatory T cells treatment and regulatory T cells therapy are used herein with the same meaning.

The term “allogeneic cells” as used herein refers to cells isolated from one subject (the donor) and infused in another (the recipient or host).

The term “autologous cells” as used herein refers to cells that are isolated and infused back into the same subject (recipient or host).

The term “polyclonal” as used herein refers to a population comprising multiple clones recognizing different epitopes of the same antigen or of different antigens.

The term “monoclonal” as used herein refers to a population comprising a single clone derived from a single cell and recognizing one epitope of a single antigen.

### DETAILED DESCRIPTION

The inventors made the surprising observation that a low dose of regulatory T cells is efficient for treating a condition in a subject in need thereof, whereas the conventional dose for cell therapy is inefficient.

Without willing to be bound by a theory, the inventors suggest that low doses of regulatory T cells are more efficient that high doses for treating diseases because low doses of regulatory T cells would be interpreted by the organism as a new debuting immune response, having the field to progress in a positive manner with full proliferation and suppressive effect.

One object of the invention is regulatory T cells for treating or for use in treating a patient in need thereof, wherein a therapeutically effective dose of 10⁴ to 10⁶ regulatory T cells is administered to the patient.

One object of the invention is regulatory T cells for treating or for use in treating a patient in need thereof, wherein a therapeutically effective dose of 1 10⁴ to 9.99 10⁵ regulatory T cells is administered to the patient.

One object of the invention is a method for treating a patient in need thereof, comprising administering to the patient a therapeutically effective dose of 10⁴ to 10⁶ regulatory T cells.

One object of the invention is a method for treating a patient in need thereof, comprising administering to the patient a therapeutically effective dose of 1 10⁴ to 9.99 10⁵ regulatory T cells.

In one embodiment, the patient is a human and the regulatory T cells to be administrated are human cells.

In one embodiment of the invention, the therapeutically effective dose to be administrated in the patient is 1 10⁵ to 9.99 10⁵ regulatory T cells.

In one embodiment of the invention, the therapeutically effective dose to be administrated in the patient is 1 10⁴, 2 10⁴, 3 10⁴, 4 10⁴, 5 10⁴, 6 10⁴, 7 10⁴, 8 10⁴, 9 10⁴, 10 10⁴ regulatory T cells.

In another embodiment of the invention, the therapeutically effective dose to be administrated in the patient is 1 10⁵, 2 10⁵, 3 10⁵, 4 10⁵, 5 10⁵, 6 10⁵, 7 10⁵, 8 10⁵, 9 10⁵, 9.99 10⁵ regulatory T cells.

Another object of the invention is regulatory T cells for treating or for use in treating a patient in need thereof, wherein a therapeutically effective dose of 1 10⁴ to 3 10⁴ regulatory cells per kg is administered to the patient.

Another object of the invention is a method for treating a patient in need thereof, comprising administering to the patient a therapeutically effective dose of 1 10⁴ to 3 10⁴ regulatory cells per kg.

In one embodiment of the invention, the therapeutically effective dose to be administrated in the patient is 1 10⁴, 1.1 10⁴, 1.2 10⁴, 1.3 10⁴, 1.4 10⁴, 1.5 10⁴, 1.6 10⁴, 1.7 10⁴, 1.8 10⁴, 1.9 10⁴, 2 10⁴, 2.1 10⁴, 2.2 10⁴, 2.3 10⁴, 2.4 10⁴, 2.5 10⁴, 2.6 10⁴, 2.7 10⁴, 2.8 10⁴, 2.9 10⁴, 3 10⁴ regulatory cells per kg.

According to the invention, the regulatory T cells to be administrated to the patient are human regulatory T cells and comprise CD4⁺CD25⁺ regulatory T cells or FoxP3⁺ regulatory T cells (natural or conventional Treg), Tr1 cells, TGF-β secreting Th3 cells, regulatory NKT cells, regulatory γδ T cells, regulatory CD8⁺ T cells, double negative regulatory T cells, in vitro induced regulatory T cells or mixture thereof.

The term “Tr1 cells” as used herein refers to cells having the following phenotype at rest CD4⁺CD25⁻FoxP3⁻ and capable of secreting high levels of IL-10 and intermediate levels of TGF-β upon activation. Tr1 cells are characterized, in part, by their unique cytokine profile: they produce high levels of IL-10, intermediate levels of TGF-β and intermediate levels of IFN-γ, but little or no IL-4 or IL-2. The cytokine production is typically evaluated in cultures of cells after activation with polyclonal activators of T lymphocytes such as anti-CD3+ anti-CD28 antibodies or Interleukin-2, PMA+ ionomycin. Alternatively, the cytokine production is evaluated in cultures of cells after activation with the specific T-cell antigen presented by antigen presenting cells. High levels of IL-10 correspond to at least about 500 pg/ml, typically greater than about 1, 2, 4, 6, 8, 10, 12, 14, 16, 18, or 20 thousand pg/ml or more. Intermediate levels of TGF-β correspond to at least about 100 pg/ml, typically greater than about 200, 300, 400, 600, 800, or 1000 pg/ml or more. Intermediate levels of IFN-γ correspond to concentrations comprised between 0 pg/ml and at least 400 pg/ml, typically greater than about 600, 800, 1000, 1200, 1400, 1600, 1800, or 2000 pg/ml or more. Little or no IL-4 or IL-2 corresponds to less than about 500 pg/ml, preferably less than about 250, 100, 75, or 50 pg/ml, or less.

The term “natural regulatory. T cells” as used herein refers to cells having the following phenotype at rest CD4⁺CD25⁺FoxP3⁺.

The term “Th3 cells” as used herein refers to cells having the following phenotype CD4⁺FoxP3⁺ and capable of secreting high levels TGF-β upon activation, amounts of IL-4 and IL-10 and no IFN-γ or IL-2. These cells are TGF-β derived.

The term “regulatory NKT cells” as used herein refers to cells having the following phenotype at rest CD161⁺CD56⁺CD16⁺ and a Vα24/Vβ11 TCR.

The term “regulatory CD8⁺ T cells” as used herein refers to cells having the following phenotype at rest CD8⁺CD122⁺ and capable of secreting highs levels of IL-10 upon activation.

The term “double negative regulatory T cells” as used herein refers to cells having the following phenotype at rest TCRαβ⁺CD4⁻CD8⁻.

The term “in vitro inducible regulatory T cells” as used herein refers to naive T cells that are differentiated into regulatory T cells in vitro.

One example of said in vitro inducible regulatory T cells is Th3 cells that are differentiated from naïve T cells in the presence of TGF-β. Other examples are natural regulatory T cells or Tr1 cells obtained by in vitro differentiation.

The term “γδ T cells” as used herein refers to T lymphocytes that express the [gamma] [delta] heterodimer of the TCR. Unlike the [alpha][beta] T lymphocytes, they recognize non-peptide antigens via a mechanism independent of presentation by MHC molecules. Two populations of γδ T cells may be described: the γδ T lymphocytes with the V γ9V δ2 receptor, which represent the majority population in peripheral blood and the γδ T lymphocytes with the V δ1 receptor, which represent the majority population in the mucosa and have only a very limited presence in peripheral blood. V γ9V δ2 T lymphocytes are known to be involved in the immune response against intracellular pathogens and hematological diseases.

In one embodiment of the invention, the regulatory T cells to be administrated to the patient are Tr1 cells.

In another embodiment of the invention, the regulatory T cells to be administrated to the patient are CD4⁺CD25⁺ regulatory T cells or FoxP3⁺ regulatory T cells (natural Treg).

In another embodiment of the invention, the regulatory T cells to be administrated to the patient are TGF-β secreting Th3 cells.

In another embodiment of the invention, the regulatory T cells to be administrated to the patient are regulatory NKT cells.

In one embodiment of the invention, the regulatory T cells to be administrated to the patient are autologous regulatory T cells or allogeneic regulatory T cells.

In one embodiment of the invention, the regulatory T cells to be administrated to the patient may be a polyclonal or a monoclonal cell population.

In another embodiment of the invention, the regulatory T cells to be administrated to the patient may be specific of an antigen or specific of multiple antigens.

In another embodiment of the invention, the regulatory T cells to be administered to the patient are natural regulatory T cells specific of multiple antigens.

In another embodiment of the invention, the regulatory T cells to be administered to the patient are natural regulatory T cells specific of an antigen.

In another embodiment of the invention, the regulatory T cells to be administered to the patient are Tr1 cells specific of an antigen.

In another embodiment of the invention, the regulatory T cells to be administered to the patient are Tr1 cells specific of multiple antigens.

Examples of antigen to which the regulatory T cells may be specific include, but are not limited to, auto-antigens; food antigen from common human diet; inflammatory antigens such as multiple sclerosis-associated antigens or joint-associated antigens; allergens and bacterial antigens.

The term “food antigen from common human diet” refers to an immunogenic peptide, which comes from foodstuffs common for humans, such as food antigens of the following non-limiting list: bovine antigens such as lipocalin, Ca-binding S100, alpha-lactalbumin, lactoglobulins such as beta-lactoglobulin, bovine serum albumin, caseins. Food-antigens may also be atlantic salmon antigens such as parvalbumin, chicken antigens such as ovomucoid, ovalbumin, Ag22, conalbumin, lysozyme or chicken serum albumin, peanuts, shrimp antigens such as tropomyosin, wheat antigens such as agglutinin or gliadin, celery antigens such as celery profilin, carrot antigens such as carrot profilin, apple antigens such as thaumatin, apple lipid transfer protein, apple profilin, pear antigens such as pear profilin, isoflavone reductase, avocado antigens such as endochitinase, apricot antigens such as apricot lipid transfer protein, peach antigens such as peach lipid transfer protein or peach profilin, soybean antigens such as HPS, soybean profilin or (SAM22) PR-10 prot.

The term “auto-antigen” refers to an immunogenic peptide derived from a protein of said individual. It may be, by way of example, an auto-antigen of the following non-limiting list: acethylcholine receptor, actin, adenin nucleotide translocator, adrenoreceptor, aromatic L-amino acid decarboxylase, asioaloglycoprotein receptor, bactericidal/permeability increasing protein (BPi), calcium sensing receptor, cholesterol side chain cleavage enzyme, collagen type IV-chain, cytochrome P450 2D6, desmin, desmoglein-1, desmoglein-3, F-actin, GM-gangliosides, glutamate decarboxylase, glutamate receptor, H/K ATPase, 17-hydroxylase, 21-hydroxylase, IA-2 (ICAS12), insulin, insulin receptor, intrinsic factor type 1, leucocyte function antigen 1, myelin associated glycoprotein, myelin basic protein, myelin oligodendrocyte protein, myosin, P80-coilin, pyruvate deshydrogenase complex E2 (PDC-E2), sodium iodide symporter, SOX-10, thyroid and eye muscle shared protein, thyroglobulin, thyroid peroxydase, thyrotropin receptor, tissue transglutaminase, transcription coactivator p75, tryptophan hydroxylase, tyrosinase, tyrosine hydroxylase, ACTH, aminoacyl-tRNA-hystidyl synthetase, cardiolipin, carbonic anhydrase II, cebtromere associated proteins, DNA-dependant nucleosome-stimulated ATPase, fibrillarin, fibronectin, glucose 6 phosphate isomerase, beta 2-glycoprotein I, golgin (95, 97, 160, 180), heat shock proteins, hemidesmosomal protein 180, histone H2A, H2B, keratin, IgE receptor, Ku-DNA protein kinase, Ku-nucleoprotein, La phosphoprotein, myeloperoxydase, proteinase 3, RNA polymerase I-III, signal recognition protein, topoisomerase I, tubulin, vimenscin, myelin associated oligodendrocyte basic protein (MOBP), proteolipid protein, oligodendrocyte specific protein (OSP/Claudin 11), cyclic nucleotide 3′phosphodiesterase (CNPase), BP antigen 1 (BPAG1-e), transaldolase (TAL), human mitochondrial autoantigens PDC-E2 (Novo 1 and 2), OGDC-E2 (Novo 3), and BCOADC-E2 (Novo 4), bullous pemphigoid (BP)180, laminin 5 (LN5), DEAD-box protein 48 (DDX48) or insulinoma-associated antigen-2.

The term “multiple sclerosis-associated antigen” refers to myelin basic protein (MBP), myelin associated glycoprotein (MAG), myelin oligodendrocyte protein (MOG), proteolipid protein (PLP), oligodendrocyte myelin oligoprotein (OMGP), myelin associated oligodendrocyte basic protein (MOBP), oligodendrocyte specific protein (OSP/Claudinl 1), heat shock proteins, oligodendrocyte specific proteins (OSP), NOGO A, glycoprotein Po, peripheral myelin protein 22 (PMP22), 2′3′-cyclic nucleotide 3″-phosphodiesterase (CNPase), fragments, variants and mixtures thereof.

The term “joint-associated antigen” refers to citrulline-substituted cyclic and linear filaggrin peptides, collagen type II peptides, human cartilage glycoprotein 39 (HCgp39) peptides, HSP, heterogenous nuclear ribonucleoprotein (hnRNP) A2 peptides, hnRNP Bl, hnRNP D, Ro60/52, HSP60, 65, 70 and 90, BiP, keratin, vimentin, fibrinogen, collagen type I, III, IV and V peptides, annexin V, Glucose 6 phosphate isomerase (GPI), acetyl-calpastatin, pyruvate deshydrogenase (PDH), aldolase, topoisomerase I, snRNP, PARP, Scl-70, Scl-100, phospholipid antigen including anionic cardiolipin and phosphatidylserine, neutrally charged phosphatidylethanolamine and phosphatidylcholine, matrix metalloproteinase, fibrillin, aggreccan.

The term “allergen” refers to an inhaled allergen, an ingested allergen or a contact allergen. Examples of allergens include, but are not limited to, inhaled allergens derived from pollens (Cup, Jun), house dust mites (Der, Gly, Tyr, Lep), dog, cat and rodents (Can, Fel, Mus, Rat). Examples of contact allergens include, but are not limited to, heavy metals (such as nickel, chrome, gold), latex, haptens such as halothane, hydralazine.

Examples of bacterial antigens include capsule antigens (e.g., protein or polysaccharide antigens such as CP5 or CP8 from the S. aureus capsule); cell wall (including outer membrane) antigens such as peptidoglycan (e.g., mucopeptides, glycopeptides, mureins, muramic acid residues, and glucose amine residues) polysaccharides, teichoic acids (e.g., ribitol teichoic acids and glycerol teichoic acids), phospholipids, hopanoids, and lipopolysaccharides (e.g., the lipid A or O-polysaccharide moieties of bacteria such as Pseudomonas aeruginosa serotype O11); plasma membrane components including phospholipids, hopanoids, and proteins; proteins and peptidoglycan found within the periplasm; fimbrae antigens, pili antigens, flagellar antigens, and S-layer antigens. S. aureus antigens can be a serotype 5 capsular antigen, a serotype 8 capsular antigen, and antigen shared by serotypes 5 and 8 capsular antigens, a serotype 336 capsular antigen, protein A, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1-6 glucosamine, catalase, beta-lactamase, teichoic acid, peptidoglycan, a penicillin binding protein, chemotaxis inhibiting protein, complement inhibitor, Sbi, and von Willebrand factor binding protein

In one embodiment of the invention, the regulatory T cells to be administrated to the patient may be obtained from blood, such as peripheral blood or umbilical cord blood, or from tissue biopsy such as lymph node biopsy, intestinal or synovial biopsies or mucosal tissue biopsy, or from bronchoalveolar lavage or a cerebrospinal fluid.

In one embodiment of the invention, the regulatory T cells to be administered to the patient are comprised in a pharmaceutical composition with a pharmaceutically acceptable carrier.

The pharmaceutically acceptable carriers useful herein are conventional. Remington's Pharmaceutical Sciences 16th edition, Osol, A. Ed. (1980) describes compositions and formulations suitable for pharmaceutical delivery of the composition of the present invention. In general, the nature of the carrier will depend on the mode of administration being employed. For instance, parenteral formulations usually comprise injectable fluids that include pharmaceutically and physiologically acceptable fluids such as water, physiological saline, balanced salt solutions, aqueous dextrose, sesame oil, glycerol, ethanol, combinations thereof, or the like, as vehicle. The carrier and composition can be sterile, and the formulation suits the mode of administration. In addition to biological neutral carriers, pharmaceutical compositions to be administrated can contain minor amounts of non toxic auxiliary substances, such as wetting or emulsifying agents, preservatives, and pH buffering agents and the like, for example sodium acetate or sorbitan monolaurate. The composition can be a liquid solution, suspension, emulsion.

In another embodiment of the invention, the composition comprising the regulatory T cells may be formulated for parenteral, intramuscular, intra-tissular, intravenous or intra-peritoneal injection, intranasal inhalation, lung inhalation, intradermal or intra-articular injection.

Preferably, the medicament or pharmaceutical composition of the invention may be administrated by intramuscular, intraperitoneal or intravenous injection, or by direct injection into the lymph nodes of the patient or directly into the inflammatory site or directly into the transplanted organ, more preferably by intravenous injection.

In one embodiment of the invention, the composition comprising the regulatory T cells to be administrated to the patient is in a pouch/infusion bag or in a syringe.

In one embodiment of the invention, the pouch/infusion bag or the syringe comprises 100 μl to 500 ml of the composition.

In another embodiment, the pouch/infusion bag or the syringe comprises from 100 μl to 100 ml of the composition.

In another embodiment, the pouch/infusion bag or the syringe comprises from 100 μl to 50 ml of the composition.

In another embodiment, the pouch/infusion bag or the syringe comprises from 100 μl to 10 ml of the composition.

In another embodiment, the pouch/infusion bag or the syringe comprises from 100 μl to 5 ml of the composition.

One object of the invention is a medical device such as a pouch/infusion bag or a syringe comprising a therapeutically effective dose as described here above of regulatory T cells or a pharmaceutical composition comprising a therapeutically effective dose as described here above of regulatory T cells.

In one embodiment of the invention, the therapeutically effective dose of regulatory T cells as described here above is administrated to the patient once a week, once every two weeks, once every 3 weeks, or once every 4 weeks. In another embodiment, the therapeutically effective dose of regulatory T cells as described here above is administrated to the patient once every month, once every two months, once every three months, once every four months, once every five months or once every six months.

In another embodiment, the therapeutically effective dose of regulatory T cells as described here above is administrated to the patient once every 8 weeks.

Hereafter are described examples of methods for obtaining autologous or allogeneic regulatory T cells.

One method for obtaining human Tr1 cells comprises:
a) isolating a progenitor cell population from a subject,
b) obtaining a population of dendritic cells by culturing said progenitor cell population in the presence of IL-10,
c) contacting cells of step b) with a CD4+T lymphocyte population isolated from said subject in the presence of an antigen, to allow differentiation of CD4+ T cells directed to said antigen into the Tr1 cell population, and
d) recovering the Tr1 cell population from the step c).

In step b), IL-10 is present from 50 to 250 U/ml, preferably at 100 U/ml in the culture medium. Said method for obtaining Tr1 cells is described in Wakkach et al (Immunity 2003 May; 18(5):605-17).

Said method may also be carried out using Dexamethasone and Vitamin D3, or tolerogenised or immature DCs instead of the DCs of step b).

Another method for obtaining human Tr1 cells comprises:

a) culturing a CD4+ T cell population directed to an antigen, isolated from a subject in a media with an appropriate amount of IFN-α, and
b) recovering the Tr1 cell population.

IFN-α is preferably present in the media at 5 ng/ml. In the step a), the media may further comprise an appropriate amount of IL-10, preferably at 100 U/ml.

In step b), the Tr1 cell population is cultured in a media comprising IL-15 to allow proliferation, IL-15 being preferably at 5 ng/ml in the media. Said method for obtaining Tr1 cells is described in the U.S. Pat. No. 6,746,670.

Another method for obtaining human Tr1 cells comprises:

a) in vitro activating a CD4+ T cell population in presence of an antigen presented by artificial antigen presenting cells, and
b) recovering an activated CD4+ T cells comprising at least 10% of Tr1 cells.

Preferably, the artificial antigen presenting cells express a HLA II system molecule and a human LFA-3 molecule and do not express the co-stimulation molecules B7-1, B7-2, B7-H1, CD40, CD23 and ICAM-1.

Said method for obtaining Tr1 cells is described in the patent application WO02/092793.

Another method for obtaining human Tr1 cells comprises:

a) in vitro activating a CD4+ T cell population in presence of an antigen and an appropriate amount of IL-10; and
b) recovering the Tr1 cell population.

Preferably, IL-10 is present in the media at 100 U/ml. Said method for obtaining Tr1 cells is described in Groux et al. (Nature 1997, 389(6652):737-42).

Another method for obtaining human Tr1 cells comprises:

a) stimulating a leukocyte population or a peripheral blood mononuclear cell (PBMC) population with an antigen,
b) recovering the antigen-specific Tr1 cell population from the stimulated population,
c) optionally expanding said antigen-specific Tr1 cell population. Said method for obtaining Tr1 cells is described in WO 2007010406.

Another method for obtaining human Tr1 cells comprises cultivating CD4+ T cells in the presence of IL-27 and TGF-b such as described in Awasthi et al. Nat. Immunol. 2007 8(12): 1380 or in Apetoh et al. Nat. Immunol 2010 11(9): 854.

Leukocytes encompass several types of cells, which are characterized by their importance, their distribution, their number, their lifetime and their potentiality. These types are the following: the polynuclear or granular leukocytes, among which one finds the eosinophilic, the neutrophilic and the basophilic leukocytes, and the mononuclear cells, or peripheral blood mononuclear cells (PBMCs), which are large white blood cells and consist in the major cell types of the immune system (lymphocytes and monocytes). The leukocytes or the PBMCs can be separated from the peripheral blood by any method known to those skilled in the art. Advantageously, for the separation of the PBMCs, centrifugation may be used, preferably density gradient centrifugation, preferably discontinuous density gradient centrifugation. An alternative is the use of specific monoclonal antibodies. In certain embodiments PBMC are typically isolated from the whole blood product by means of Ficoll-Hypaque, using standard procedures. In other embodiments the PBMCs are recovered by means of leukapheresis. Said method is described in the patent application WO2007/010406.

Another method for obtaining human Tr1 cells comprises:

a) culturing a leukocyte population or a peripheral blood mononuclear cell (PBMC) population with mesenchymal stem cells in the presence of antigen,
b) recovering the Tr1 cell population.

Said method can also be carried out with naïve or memory T cells instead of PBMC or leukocytes.

The Tr1 cell population thus obtained may further be expanded by culture in presence of cytokines such as Interleukin-2 and Interleukin-4. Alternatively, Interleukin-15 and Interleukin-13 could also be used in Tr1 cell expansion cultures.

Tr1 cells can be identified and/or purified by Elisa, flow cytometry, or immunoaffinity methods with antibodies directed against markers including CD4⁺, CD11a⁺, CD18⁺, PSGL-1^{+/-}, IL-10.

Tr1 cells can also be enriched by positive selection or negative selection using flow cytometry or magnetic beads. Such methods are also described in WO2005/000344.

One method for expanding in vitro Tr1 cells is described in WO2006/108882. Said method comprises:

a) cultivating at a temperature T1 inferior to 35° C., in a culture medium Mf, feeder cells such as insect feeder cells, said temperature T1 allowing the proliferation of feeder cells and said feeder cells expressing factors which interact with the following cell surface proteins:
the CD3/TCR complex,
the CD28 protein,
the IL-2 receptor,
the CD2 protein,
the IL-4 receptor,
   b) contacting the feeder cells obtained in step a) cleared or not of their culture medium Mf, with the Tr1 cell population contained in the culture medium Mp, wherein said culture medium Mp does not initially contain the factors cited in step a), in order to obtain a mixture containing the Tr1 cell population, the feeder cells and the culture medium Mp,
   c) cultivating the mixture obtained at step b) at a temperature T2 which is at least 35° C., said temperature being chosen such that the Tr1 cell population proliferates and the feeder cells do not proliferate,
   d) recovering the Tr1 cell population such expanded.

Examples of factors which interact with the above mentioned cell surface proteins include
an anti-CD3 monoclonal antibody or a modified anti-CD3 antibody, wherein the anti-CD3 intracytoplasmic domain of the CD3 heavy chain is replaced with a transmembrane domain,
the CD80 or CD86 protein,
the IL-2 secreted by the feeder cells,
the CD58 protein,
an interleukin selected from the group comprising IL-4 and IL-13.

An anti-CD3 monoclonal antibody can be used to activate a population of T cells via the TCR/CD3 complex, advantageously a modified anti-CD3 antibody, wherein the modification of the anti-CD3 antibody consists in the replacement of the intracytoplasmic domain with a transmembrane domain, such that said modified anti-CD3 antibody anchors to the cellular membrane of the feeder cells and interacts with the CD3/TCR protein complex of the T cells. The factor interacting with the CD28 protein present at the surface of the antigen-specific Tr1 cells and which is expressed by the feeder cells, may be an anti-CD28 monoclonal antibody or a fragment thereof capable of crosslinking the CD28 molecule; in such a case, modification of the anti-CD28 monoclonal antibody can be envisaged by adding a transmembrane domain in order that it anchors to the cell surface of the feeder cells. Preferably, the natural ligand for CD28 is employed instead of the anti-CD28 monoclonal antibody, that is to say for example a member of the B7 family of proteins, such as B7-1(CD80) and B7-2 (CD86) proteins.

The factor expressed by the feeder cells which interacts with CD2 may be an anti-CD2 monoclonal antibody or a fragment thereof capable of crosslinking the CD2 molecule; modification of the anti-CD2 monoclonal antibody can be envisaged by adding a transmembrane domain for anchoring to the cell surface of the feeder cells. Preferably, the natural ligand for CD2 is employed instead of the anti-CD2 monoclonal antibody, that is to say the CD58 protein.

In addition to the factors which are anchored to the cell membrane of the feeder cells, factors which are secreted, such as interleukins, are also required for expansion of the antigen-specific Tr1 cell population. Among these interleukins are the IL-2, which interacts with the IL-2 receptor present at the surface of the antigen-specific Tr1 cells, and either the IL-4 or the IL-13, which interacts with the IL-4 receptor of the antigen-specific Tr1 cells.

Another method for expanding Tr1 cells comprises culturing Tr1 cells with anti-CD3/28 beads in the presence of cytokines such as IL-2, IL-4, IL-13 and/or IL-15.

One method for isolating natural regulatory T cells comprises using flow cytometry to sort natural regulatory T cells based on a combination of markers including CD4⁺, CD25⁺ and CD127^{low/-}. This method leads to a highly enriched cell population that is >95% FoxP3⁺.

Another method for isolating natural regulatory T cells comprises using flow cytometry to sort natural regulatory T cells based on a combination of markers including CD45RA⁺, CD4⁺ and CD25⁺. Said method is described in US2010/291678.

One method for expanding natural regulatory T cells is also described in US2010/291678 and uses anti-CD3/28 monoclonal antibody (mAb) coated beads in combination with IL-2 and irradiated feeder cells.

Another method for obtaining natural regulatory T cells comprises using flow cytometry to sort natural regulatory T cells based on CD25 expression and expand them by:
culturing them with autologous monocyte-derived dendritic cells at a 10:1 ratio of T cells:DCs in the presence of IL-2 (10 U/l), or
culturing them with rapamycin.

Another method for obtaining natural regulatory T cells comprises culturing CD4+CD25− T cells in the presence of TGF-β with an anti-CD3/CD28 stimulation for 5 days.

One method for isolating regulatory NK T cells comprises using the αGalCer-loaded CD1d multimers.

Another method for isolating regulatory NK T cells comprises using the 6B11 monoclonal antibody.

Another method for isolating regulatory NK T cells comprises using an antibody staining for Vα24 and Vβ11 or an antibody staining for Vα24.

One method for obtaining regulatory Th3 cells comprises culturing CD4⁺ T cells in the presence of TGF-β with anti-CD3/28 stimulation.

One method for expanding γδ T cells in vitro comprises starting from PBMCs by stimulation with phosphorylated compounds of bacterial origin containing nucleotides or by means of isoprenoid pyrophosphates such as isopentenyl pyrophosphate (IPP) in the presence of cytokines, such as IL-2, IL-15 and TGF-β (see WO 03/070921, WO 2009037723 for example).

According to the invention, the above described regulatory T cells are for treating a patient having an autoimmune disease, an inflammatory condition, an allergic or asthmatic condition, graft versus host disease or undergoing a transplantation.

According to the invention, the above described method is for treating a patient having an autoimmune disease, an inflammatory condition, an allergic or asthmatic condition, graft versus host disease or undergoing a transplantation.

In one embodiment of the invention, the transplantation may be a hematopoietic stem cell transplantation or a solid organ (liver, kidney, lung, heart . . . ) transplantation.

In another embodiment of the invention, examples of autoimmune diseases include, but are not limited to, diabetes, multiple sclerosis and arthritic condition.

“Arthritic condition” refers to rheumatoid arthritis, polychondritis, septic arthritis, spondyloarthropathies or, ankylosing spondylitis, juvenile idiopathic arthritis (JIA), psoriatic arthritis and diseases associated with arthritis such as systemic lupus erythematous, Sjogren's syndrome, scleroderma, dermatomyosotis, polymyosotis, polymyalgia rheumatica, fibromyalgia, sarcoidosis, vasculitis.

In another embodiment of the invention, examples of inflammatory condition include, but are not limited to, inflammatory bowel disease, ulcerative colitis, Crohn's disease, intestinal inflammation linked to food allergy or intolerance, intestinal inflammation linked to milk protein allergy, intestinal inflammation linked to celiac disease, intestinal inflammation linked to hen egg allergy, or intestinal inflammation linked to peanut allergy.

In another embodiment of the invention, examples of allergic or asthmatic condition include, but are not limited to, asthma, atopic dermatitis, allergic rhinitis, conjunctivitis, eczema, contact allergy, inhaled allergy, ingested allergy and anaphylaxis.

In one embodiment of the invention, a blood sample from the subject to be treated is collected.

Tr1 cells specific for a selected antigen are obtained by culturing PBMC with the selected antigen for 7 days. Cytokines such as IL-2 and IL-4 may optionally be added at day 3 to the culture.

The Tr1 cells obtained are then cloned by conventional methods and further expanded. Preferably, expansion of Tr1 clones directed to a selected antigen is carried out with the following method described here above:

a) cultivating at a temperature T1 inferior to 35° C., in a culture medium Mf, feeder cells such as insect feeder cells, said temperature T1 allowing the proliferation of feeder cells and said feeder cells expressing factors which interact with the following cell surface proteins:
the CD3/TCR complex,
the CD28 protein,
the IL-2 receptor,
the CD2 protein,
the IL-4 receptor,
   b) contacting the feeder cells obtained in step a) cleared or not of their culture medium Mf, with the Tr1 cell population contained in the culture medium Mp, wherein said culture medium Mp does not initially contain the factors cited in step a), in order to obtain a mixture containing the Tr1 cell population, the feeder cells and the culture medium Mp,
   c) cultivating the mixture obtained at step b) at a temperature T2 which is at least 35° C., said temperature being chosen such that the Tr1 cell population proliferates and the feeder cells do not proliferate,
   d) recovering the Tr1 cell population such expanded.

An effective dose comprising 10⁴ to 10⁶ Tr1 cells specific for a selected antigen is finally prepared and re-infused in the patient.

In one embodiment of the invention, the regulatory T cells to be administrated to a patient for treating an intestinal inflammatory condition are Tr1 cells specific for a food antigen from the common human diet.

In another embodiment of the invention, said Tr1 cells are specific for ovalbumin and are intended for treating inflammatory bowel disease, ulcerative colitis, Crohn's disease, intestinal inflammation linked to food allergy or intolerance, intestinal inflammation linked to milk protein allergy, intestinal inflammation linked to celiac disease, intestinal inflammation linked to hen egg allergy, or intestinal inflammation linked to peanut allergy.

In one embodiment of the invention, the regulatory T cells to be administrated to a patient for treating a multiple sclerosis condition are Tr1 cells specific for a multiple sclerosis associated antigen.

In another embodiment of the invention, said Tr1 cells are specific for MBP or MOG and are intended for treating multiple sclerosis.

In one embodiment of the invention, the regulatory T cells to be administrated to a patient for treating an arthritic condition are Tr1 cells specific for a joint-associated antigen.

In another embodiment of the invention, said Tr1 cells are specific for type II collagen or HSP antigen and are intended for treating rheumatoid arthritis, polychondritis, septic arthritis, spondyloarthropathies or ankylosing spondylitis, juvenile idiopathic arthritis (JIA), psoriatic arthritis and diseases associated with arthritis such as systemic lupus erythematous, Sjogren's syndrome, scleroderma, dermatomyosotis, polymyosotis, polymyalgia rheumatica, fibromyalgia, sarcoidosis, vasculitis.

In one embodiment of the invention, the regulatory T cells to be administrated to a patient for treating an allergic or asthmatic condition are Tr1 cells specific for an allergen associated with said allergic or asthmatic condition.

In another embodiment of the invention, said Tr1 cells are specific for allergens derived from pollens (Cup, Jun), house dust mites (Der, Gly, Tyr, Lep), dog, cat and rodents (Can, Fel, Mus, Rat) and are intended for treating asthma, atopic dermatitis, allergic rhinitis, conjunctivitis, eczema and anaphylaxis.

### BRIEF DESCRIPTION OF THE DRAWINGS

FIG. 1: CDAI individual responses to treatment after 5 weeks (A) and 8 weeks (B).

FIG. 2: CDAI cohort responses to treatment after 5 and 8 weeks (A) and IBDQ cohort response to treatment after 8 weeks (B).

FIG. 3: Percentage of response (A) and remission (B).

FIG. 4: (A) in vitro proliferative response of PBMC of responders to ovalbumin. (B) Percentage of decrease of proliferation to ovalbumin by cohorts.

FIG. 5: CDAI individual response in Crohn's disease patients infused twice with ovalbumin specific Tr1 cells at different dosages. R=Response; NR=Non Response.

FIG. 6: CDAI individual response in Crohn's disease Patients during 8 weeks after cell infusion. Black circles: 10⁶ dose; white squares: 10⁹ dose.

### EXAMPLES

### Experimental Procedures

### Ovalbumin Specific Tr1 Clone Production

Ovalbumin specific Tr1 clones were produced from Peripheral Blood Mononuclear Cells (PBMC) of Crohn's Disease patients. After PBMC isolation by Ficoll gradient density centrifugation (GE Healthcare, Uppsala, Sweden), cells were cultured in the presence of native irradiated ovalbumin (Sigma Aldrich, St-Louis, Mo., USA) in X-Vivo15 (Cambrex, East Rutherford, N.J.) and cytokine-enriched Drosophila feeder cell supernatants at 37° C., 5% CO2. After several days of culture, cells are cloned by limiting dilution method on layers of Drosophila feeder cells in X-Vivo15 at 37° C., 5% CO2. Growing clones are then harvested and tested for antigen specificity and Tr1 cell identity before being expanded on Drosophila feeder cells up to 5 billions.

### Drosophila Feeder Cells

Drosophila feeder cells were engineered by TxCell in order to improve the stimulation and growth of Tr1 cell clones. Schneider 2 Drosophila cells were transfected with a transmembrane form of a murine anti-human CD3 antibody, with human CD80, human CD58, human IL-2 and human IL-4. Cells are grown routinely in Express five medium from PAA laboratories (Pashing, Austria).

### Tr1 Cell Treatment of Crohn's Disease Patients

A phase I/IIa clinical trial was carried out to evaluate the tolerability of Tr1 treatment has started in severe refractory Crohn's disease patients. 4 doses of 10⁶, 10⁷, 10⁸ and 10⁹ autologous ovalbumin specific Tr1 cells were infused intravenously to the patients at a time when the CDAI (Crohn's Disease Activity Index, see below for description) is above 220 confirming an active disease. Patients where then monitored during 12 weeks for their disease activity.

### Clinical Response Assessment

The Crohn's Disease Activity Index or CDAI is a research tool used to quantify the disease activity of patients with Crohn's disease. This is of importance in research studies done on medications used to treat Crohn's disease; most major studies on newer medications use the CDAI in order to define response or remission of disease. A score of more than 220 identifies a patient with active pathology; a CDAI lower or equal to 150 identifies a patient in remission of the disease. A diminution of 100 points of CDAI after patient treatment compared to baseline (CDAI taken before treatment) is considered as a response to treatment.

The CDAI is calculated at week 0 (the week before infusion) and 1, 2, 3, 5, and 8 weeks after Tr1 cell infusion.

CDAI calculator

| | Weighting |
|---|---|
| Clinical or laboratory variable | factor |
| Number of liquid or soft stools each day for seven days | X2 |
| Abdominal pain (graded from 0-3 on severity) each day | X5 |
| for seven days | |
| General well being, subjectively assessed from 0 (well) | X7 |
| to 4 (terrible) each day for seven days | |
| Presence of complications* | X20 |
| Taking Lomitil or opiates for diarrhea | X30 |
| Presence of an abdominal mass (0 as none, 2 as questionable, | X10 |
| 5 as definite) | |
| Absolute deviation of Hematocrit from 47% in men and 42% | X6 |
| in women | |
| Percentage deviation from standard weight | X1 |

*Complications: arthralgia, uveitis, erythema nodosum, aphthous ulcers, pyoderma gangrenosum, anal fissure, new fistula, abscess (score 1 per item).

The Inflammatory Bowel Disease Questionnaire or IBDQ is another research tool used to quantify the disease activity of patients with Crohn's disease.

The Inflammatory Bowel Disease Questionnaire (IBDQ) was developed to incorporate elements of social, systemic and emotional symptoms, as well as bowel related symptoms into an activity index.

An IBDQ score of more than 170 identifies a patient in remission of the disease. An increase of at least 16 points after patient treatment compared to baseline (IBDQ determined before treatment) is considered as a response to treatment.

### Cell Culture and Proliferation Assessment

At week 0 (the week before infusion) and 1, 3, 5 and 8 weeks after Tr1 cell infusion, patient's peripheral blood was collected and PBMCs were isolated by Ficoll gradient Density centrifugation. Cells were then cultured at 10⁶ cells/ml in the presence or absence of ovalbumin (400 ng/ml) in XVivo15 medium during 5 days at 37° C., 5% CO2. After these five days culture, proliferation of the incubated cells was measured using the WST1 Kit from Roche that allows evaluating the number of viable cells per culture well.

### Results

The clinical trial described here aimed at determining the safety and efficacy of a single intravenous administration of autologous ovalbumin-specific Tr1 cells in Crohn's Disease patients with active disease (CDAI above 220).

21 patients suffering from Crohn's Disease were treated with 10⁶, 10⁷, 10⁸ or 10⁹ autologous ovalbumin-specific Tr1 cells.

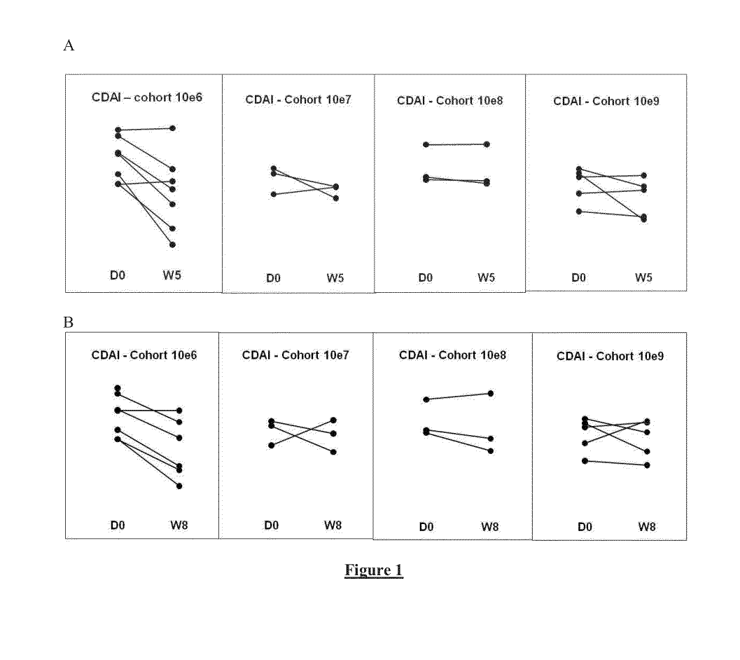

FIG. 1 shows the evolution of the CDAI of the patients between D0 (before regulatory T cell therapy) and week 5 (FIG. 1A) or week 8 (FIG. 1B). Results show that almost all patients treated with 10⁶ cells had a decrease of their CDAI, whereas less patients treated with the higher doses showed a CDAI decrease.

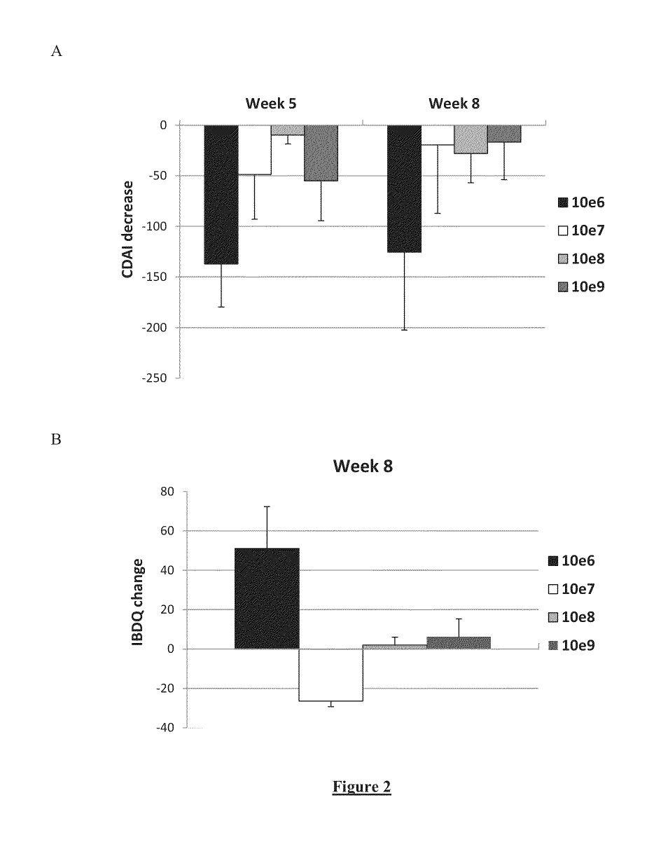

FIG. 2 shows the cohort responses to the treatment: the group of patients treated with 10⁶ cells showed a CDAI decrease of almost 150 points at week 5 and 8, whereas the groups of patients treated with the higher doses showed a CDAI decrease of less than 50 points (FIG. 2A).

Analysis of the IBDQ score at week 8 showed that the score of the group of patients treated with 10⁶ cells increased of more than 30 points, whereas the score of the group of patients treated with the higher doses did not increase or increased of less than 10 points (FIG. 2B). These results demonstrate that only the group of patients treated with 10⁶ cells responded to the treatment when analyzing the CDAI and the IBDQ scores.

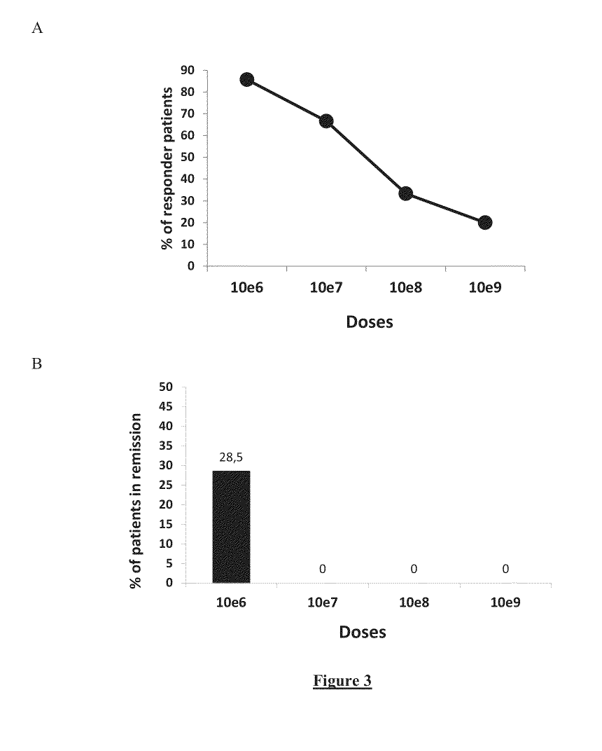

FIG. 3A shows the percentage of patients that responded to the treatment in each group: almost all patients responded to the treatment when treated with the dose of 10⁶ cells, whereas less than 20% of patients responded to the treatment when treated with the dose of 10⁹ cells.

FIG. 3B shows the percentage of patients in remission: almost 30% of patients treated with the dose of 10⁶ cells are in remission, whereas no patient treated with the higher doses is in remission.

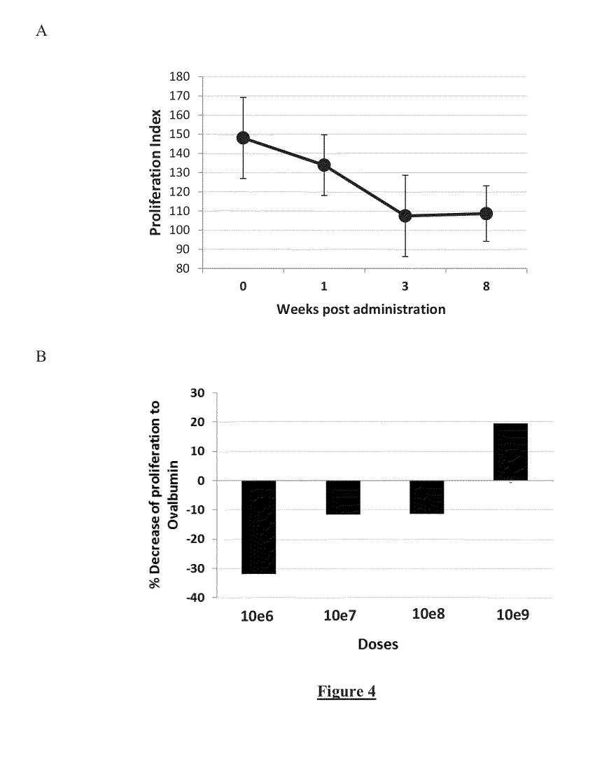

FIG. 4 shows the in vitro proliferation of PBMC to ovalbumin in responder patients. A decrease in the proliferation of PBMC to ovalbumin corresponds to an efficient action of the regulatory T cells infused in the patients.

FIG. 4A shows the in vitro proliferation of PBMC is significantly decreased at week 3 and week 8 compared to week 0 (before treatment).

FIG. 4B shows the decrease of proliferation of PBMC to ovalbumin in each group of responders: patients treated with the 10⁶ dose demonstrated a decrease of more than 30%, whereas patients treated with the 10⁷ and 10⁸ doses demonstrated a decrease of 10% and patients treated with the highest dose demonstrated no decrease in proliferation.

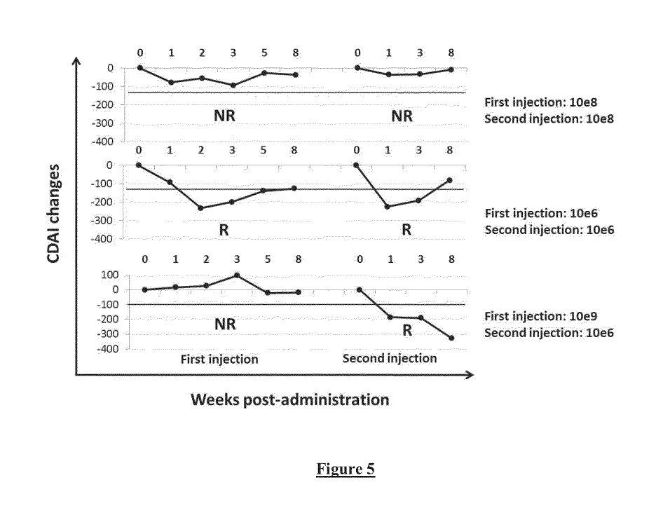

FIG. 5 confirms that only the patients treated with a 10⁶ dose of Tr1 cells are capable of inducing a CDAI decrease of more than 100 points; whereas administration of 10⁸ and 10⁹ Tr1 cells to patients had minor effects on the CDAI.

In addition, FIG. 5 shows that patients treated with a non-efficient dose such as a 10⁹ dose are capable of inducing a response to treatment after a second injection of a 10⁶ dose of Tr1 cells.

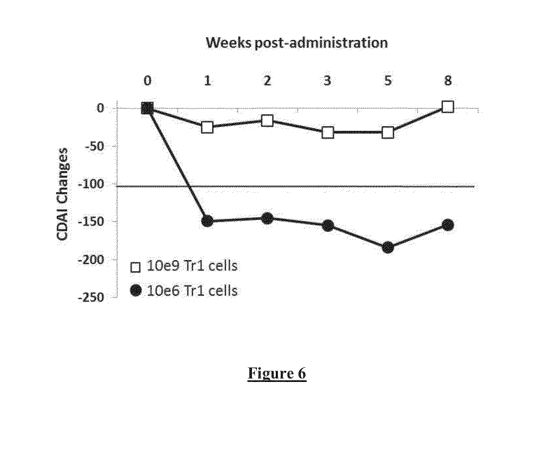

FIG. 6 confirms in two additional patients that the 10⁶ dose (black circles) induces a stable response to treatment (decrease of a least 100 points of CDAI) during the 8 weeks follow-up whereas the 10⁹ dose (white squares) had no effect on the CDAI.

Results show that response to treatment is significant in patients treated with 10⁶ cells at week 5 and week 8 after Tr1 cell administration compared to baseline (the week before Tr1 cell treatment) whereas no statistical significance is observed with the 10⁹ dose.

Statistical T-test analysis on the response to treatment at week 5 and week 8 after Tr1 cell administration for 8 patients treated at 10⁶ doses and 6 patients treated with 10⁹ cells.

TABLE 1

| Dose of cell | T-test p value W5 | T-test p value W8 | Number of |
|---|---|---|---|
| injected | compared to baseline | compared to baseline | patients |
| 10⁶ | 0.0062* | 0.0042* | 8 |
| 10⁹ | 0.1455 | 0.4987 | 6 |

T-test p value
>0.05 is not considered statistically significant
<0.05 is considered statistically significant
*<0.01 is considered highly statistically significant

1-9. (canceled)

10. A method for treating an inflammatory or an autoimmune condition in a patient in need thereof, comprising administering to the patient regulatory T cells in a therapeutically effective dose of 10⁴ to 10⁶ cells.

11. The method according to claim 10, wherein the regulatory T cells are autologous.

12. The method according to claim 10, wherein the regulatory T cells are allogeneic.

13. The method according to claim 10, wherein the regulatory T cells are polyclonal.

14. The method according to claim 10, wherein the regulatory T cells are monoclonal.

15. The method according to claim 10, wherein the regulatory T cells are specific for a single antigen.

16. The method according to claim 10, wherein the regulatory T cells are specific for multiple antigens.

17. The method according to claim 10, wherein the patient to be treated is suffering from a graft-versus-host disease or is undergoing a transplantation.

18. The method according to claim 10, wherein the patient to be treated is suffering from diabetes, multiple sclerosis, arthritic condition, inflammatory bowel disease, ulcerative colitis, Crohn's disease, or an allergic or asthmatic condition.

19. A composition comprising a therapeutically effective dose of 10⁴ to 10⁶ human regulatory T cells.

20. The composition according to claim 19, wherein the regulatory T cells are autologous.

21. The composition according to claim 19, wherein the regulatory T cells are allogeneic.

22. The composition according to claim 19, wherein the regulatory T cells are polyclonal.

23. The composition according to claim 19, wherein the regulatory T cells are monoclonal.

24. The composition according to claim 19, wherein the regulatory T cells are specific for a single antigen.

25. The composition according to claim 19, wherein the regulatory T cells are specific for multiple antigens.

26. A medical device comprising a therapeutically effective dose of 10⁴ to 10⁶ human regulatory T cells.

27. The medical device according to claim 26, wherein the regulatory T cells are autologous.

28. The medical device according to claim 26, wherein the regulatory T cells are allogeneic.

29. The medical device according to claim 26, wherein the regulatory T cells are polyclonal.

30. The medical device according to claim 26, wherein the regulatory T cells are monoclonal.

31. The medical device according to claim 26, wherein the regulatory T cells are specific for a single antigen.

32. The medical device according to claim 26, wherein the regulatory T cells are specific for multiple antigens.

33. The medical device according to claim 26, being in the form of a pouch or infusion bag or a syringe.
